(19) United States
(12) Patent Application Publication
(10) Pub. No.: US 2018/0161477 A1
(43) Pub. Date: Jun. 14, 2018

(54) Structured Mineral Bone Replacement Element

(71) Applicant: Innotere GmbH, Radebeul (DE)

(72) Inventor: Berthold Nies, Fränkisch-Crumbach (DE)

(21) Appl. No.: 15/823,627

(22) Filed: Nov. 28, 2017

(30) Foreign Application Priority Data
Dec. 8, 2016 (DE) 102016224453.1

(51) Int. Cl. A61L 27/02; A61L 24/00; A61L 24/02

(52) U.S. Cl. CPC A61L 27/02; A61L 24/0036; A61L 27/54; A61L 24/0042; A61L 24/02

(57) ABSTRACT

Structured mineral bone substitute moldings having a defined interconnecting pore system and a predetermined structure, to a method for producing structured mineral bone substitute moldings using 3D printing, and to the use thereof for producing an alloplastic implant or as a carrier material in cell culturing, tissue culturing and/or tissue engineering.

### CROSS REFERENCE TO RELATED APPLICATIONS

This application claims the priority of DE 102016224453.1 filed on 2016 Dec. 8; this application is incorporated by reference herein in its entirety.

### BACKGROUND

The invention relates to structured mineral bone substitute moldings having a defined interconnecting pore system and a predetermined structure, to a method for producing structured mineral bone substitute moldings by way of 3D printing, and to the use thereof for producing an alloplastic implant or as a carrier material in cell culturing, tissue culturing and/or tissue engineering.

Research into the development of bioresorbable implant materials in order to circumvent complex surgeries to remove inserted implants has been conducted in the field of regenerative medicine for many years now. Bioresorbable materials have the ability to gradually degrade as part of natural metabolic processes following implantation in the body as a result of enzymatic or cellular mechanisms. The resultant degradation products are largely resorbed by the body.

Conventional solutions for providing alloplastic implant materials, i.e. synthetic implant materials foreign to the body, are usually based on calcium phosphates, which are produced by way of precipitation or high temperature processes (sintering of suitable starting materials at temperatures above 500° C.). While the production by way of high-temperature processes yields materials that are similar to bone materials in terms of substance, this results in considerable changes to the structure, thus impairing the bioactivity and resorbability of a bone substitute molding.

Mineral bone cements, which harden in situ by means of a hydraulic setting reaction, i.e. a reaction with water, offer one possible alternative.

U.S. Pat. No. 6,642,285 B1 discloses a hydraulic cement for producing bone implants from three components that are matched to one another and, after mixing, are intended to harden as quickly as possible to form a macroporous solid matter. The first component represents a calcium source, which, in conjunction with an aqueous solution and a hydrophobic liquid, preferably hardens fully within 60 minutes, wherein the product, after hardening, loses its hydraulic activity, i.e. it does not react further with water.

WO 2005/084 726 A1 discloses a hydraulic bone cement, comprising a first component, this being powdery calcium phosphate particles, and a second component, consisting of water. When the two components are mixed, the composition sets particularly quickly within 1 to 20 minutes to form a solid calcium phosphate bone cement.

WO 2008 148 878 A2 discloses pastes, suspensions or dispersions having a liquid to pasty consistency, consisting of a resorbable mineral bone cement component and a carrier liquid, which are substantially anhydrous, for use as bone cements or bone substitute materials, wherein the entire setting reaction preferably takes place after implantation.

DE 10 2013 221 575 B3 discloses dimensionally stable bone substitute moldings made of mineral bone cement having residual hydraulic activity, a method for the production thereof, preferably by way of (3D) printing, and the use thereof as an alloplastic bone implant. Moreover, strontium ions are disclosed as a possible additive to the reactive mineral bone cement component.

Hofmann et al. discloses calcium phosphate cements as carvable and mechanically stable bone substitute materials, and a method for producing the calcium phosphate bone substitute material, comprising the steps of mixing a mechanically activated α-tricalcium phosphate or a mixture of tetracalcium phosphate and dicalcium phosphate anhydrate with water, shaping said mixture in a casting mold and setting it at 37° C. for 1 to 4.5 hours (Hofmann et al. 2007). The setting reaction is terminated by way of extraction in ethanol or by way of freeze drying, and complete setting takes place after implantation in the presence of water or serum.

Surprisingly, it has been found that moldings thus produced have particularly good mechanical properties when hardening does not take place in aqueous solutions but in a saturated steam atmosphere at ambient temperatures or slightly elevated temperatures (25 to 75° C.), but in particular below the sintering temperature.

WO 2012/101 428 A1 discloses a method for producing monetite from a solid component, consisting of a calcium salt, such as α-tricalcium phosphate, and an aqueous component having a monovalent or divalent metal cation and a halide anion at a temperature of 37° C., preferably in a time period of 36 hours to 30 minutes. Furthermore, WO 2012/101 428 A1 discloses the use of said monetite as bone substitute material.

Common methods for producing porous bone substitute materials, however, have little control over the arrangement of the pores and the interconnectivity thereof. Both are extremely important for rapid and lasting osseointegration of the bone substitute material—an important clinical objective.

3D printing offers a possible alternative, making new technical avenues possible for forming defined pore structures in porous bone substitute materials.

Lode et al. (Lode et al. 2012) disclose that anhydrous, pasty preparations made of mineral bone cements can be printed by way of a printing process to form porous moldings, which can subsequently be hardened to form solid moldings by placing these in aqueous solutions.

DE102013221575B3 discloses dimensionally stable bone substitute moldings having hydraulic activity by using carrier liquids having low solubility in water for mixing a moldable bone cement compound, and shaping preferably by way of 3D printing or a granulation process. In the bone substitute moldings having residual hydraulic activity, part of the setting reaction of the cement-type mineral preparation takes place under biological conditions after implantation. In this way, higher bioactivity can be achieved due to the formation of a bone-like structure.

3D printing is furthermore used in what is known as drop-on-powder printing. In this method, thin layers of calcium phosphate powder mixtures are spread and brought into contact with reactive solutions (setting liquid) in the areas that are to be solidified. This results in the locally delimited solidification of the powder bed, and repeated doctoring and contact lead to the formation of 3D structures. The non-solidified powder forms the pore system and is removed, for example by blowing it out with compressed air. Afterwards, the pre-solidified scaffold is converted into a ceramic molding by means of a sintering step at temperatures above 500° C., resulting in low flexibility of the composition of the substances to be printed due to the transformation or destruction in the final sintering process. Typical end products are moldings made of hydroxylapatite, tricalcium phosphate or biphasic calcium phosphate. The disadvantages of this method are the low specific surface area of less than 1 m²/g and the bioactivity limited thereby (due to the necessary sintering step), the small dimensions of the moldings produced and the narrow limitation of the pore distribution due to purification problems of non-solidified powder, which worsen with size and complexity.

### SUMMARY

The invention relates to structured mineral bone substitute moldings having a defined interconnecting pore system and a predetermined structure, to a method for producing structured mineral bone substitute moldings using 3D printing, and to the use thereof for producing an alloplastic implant or as a carrier material in cell culturing, tissue culturing and/or tissue engineering.

### DETAILED DESCRIPTION

It is therefore the object of the present invention to provide bone substitute moldings that are produced without a ceramic sintering step, and the shaping of which takes place under controlled conditions.

It is furthermore an object of the invention to provide bone substitute moldings that stimulate bone growth and have sufficient mechanical stability.

According to the invention, the object is achieved by the structured mineral bone substitute moldings having a defined interconnecting pore system and a predetermined structure, which are made of anhydrous mineral bone cement containing calcium and/or magnesium compounds and from 0.5 mol. % to 25 mol. % strontium ions, based on the total content of divalent cations,

wherein the defined interconnecting pore system, in at least one dimension, has an interconnecting pore system having pores that have an average pore cross section of at least 50,000 μm² and an average pore diameter of at least 250 μm, based on a circular cross section.

According to the invention, a “defined interconnecting pore system” is understood to mean a contiguous pore system that, in contrast to a statistical pore system, has a defined pattern. “Interconnecting” is understood to mean that the pores are connected to one another, wherein a cross section of at least 50,000 μm² is present at the point where two pores connect.

According to the invention, a “predetermined structure” is understood to mean a macroscopic shape, which may assume designed, arbitrary and complex geometries by way of 3D printing.

In one embodiment, the structured mineral bone substitute moldings are composed of multiple consecutive layers (as stacks), which can assume various geometric shapes, such as circular, oval or angular.

In one embodiment, the predetermined structures are geometric shapes such as cylinders, rectangles, wedges, disks, or cones or shapes anatomically adapted to the bone, as is known from cages for the spinal column. “Cages” for the spinal column are understood to mean mechanically formed space holders for between the vertebrae, which restore the natural (physiological) height of the intervertebral disk segment.

According to the invention, the defined interconnecting pore system, at least in one dimension, has an interconnecting pore system having pores that have an average pore cross section of at least 50,000 μm² and an average pore diameter of at least 250 μm, based on a circular cross section.

According to the invention, a “dimension” is understood to mean the physical measurement of a body. The dimension can be selected from the length, width or height.

Advantageously, specific pore arrangements and specific pore volumes can be established by way of 3D printing. In particular, this advantageously makes the design of directed pores possible, whereby the path for the new bone growing in to bridge a bone defect can be minimized.

In another embodiment, the defined interconnecting pore system, at least in two dimensions, has an interconnecting pore system having pores that have an average pore cross section of at least 50,000 μm² and an average pore diameter of at least 250 μm, based on a circular cross section.

In another embodiment, the defined interconnecting pore system, in three dimensions, has an interconnecting pore system having pores that have an average pore cross section of at least 50,000 μm² and an average pore diameter of at least 250 μm, based on a circular cross section.

In one embodiment, a maximum of 10% of the pores of the defined interconnecting pore system have an average pore cross section and an average pore diameter of from 50 to 100% of the average pore cross section of at least 50,000 μm² and of the average pore diameter of at least 250 μm, based on a circular cross section of the pores.

In a preferred embodiment, the defined interconnecting pore system, in at least one dimension, preferably in at least two dimensions, and particularly preferably in three dimensions, has an interconnecting pore system having pores that have an average pore cross section of at least 75,000 μm² and an average pore diameter of at least 300 μm, based on a circular cross section.

According to the invention, the defined interconnecting pore system, in at least one dimension, preferably in at least two dimensions, and particularly preferably in three dimensions, has an interconnecting pore system having pores that have a maximum average pore cross section of 785,000 μm² and a maximum average pore diameter of 1000 μm, based on a circular cross section.

By increasing the average pore cross section and the average pore diameter, the mechanical properties, in particular the compressive strength, of the structured mineral bone substitute moldings according to the invention are decreased.

In another embodiment, the structured mineral bone substitute moldings according to the invention have a total porosity in the range of from 50 to 85%, and preferably from 60 to 75%.

“Total porosity” is understood to mean the sum of macroporosity, microporosity and nanoporosity. The total porosity is derived from the weight ratio of the hardened moldings to a molding having the theoretical density of calcium phosphates of 3.0 g/cm³. “Macroporosity” is understood to mean the sum of the visible pores having a pore diameter of d>100 μm, and “microporosity” and “nanoporosity” are understood to mean the sum of the microscopically visible pores having a pore diameter of d<10 μm. Macroporosity results from shaping the bone cement compound and influences the growth of cells and tissue structures into the bone substitute moldings. Microporosity and nanoporosity result from the density of the bone cement compound and influence the specific surface area, this relating to the adsorption of biomolecules and the interaction with bone cells.

In another embodiment, the structured mineral bone substitute moldings according to the invention have a macroporosity in the range of between 30 and 65%, and a microporosity in the range of between 20 and 35%.

In another embodiment, the structured mineral bone substitute moldings according to the invention have a specific surface area of at least 5 m²/g, and preferably of between 5 and 50 m²/g.

The specific surface area of the structured mineral bone substitute moldings according to the invention can be determined by way of BET measurement and mercury porosimetry.

A “BET (Brunauer-Emmett-Teller) measurement” is understood to mean an analytical method for determining surface areas using the gas adsorption technique, in which the mass-based specific surface area is calculated from experimental data.

“Mercury porosimetry” is understood to mean an analytical method for determining surface areas using a non-wetting liquid, such as mercury. The pore size is measured as a function of the external pressure needed to force the liquid into a pore against the opposing force of the liquid's surface tension and can be described by the Washburn equation (Washburn, 1921). The pore size distribution is calculated from what are known as intrusion and extrusion curves.

According to the invention, an “anhydrous mineral bone cement” is understood to mean a mineral bone cement that is mixed without adding water. A “reactive mineral bone cement” is understood to mean the at least one reactive mineral or organomineral solid component (bone cement component) that is able to set in a setting process so as to form a poorly soluble solid when brought into contact with an aqueous solution or after being introduced into an aqueous solution.

Mineral bone cements consist of at least one reactive mineral or organomineral solid component (bone cement component) that is able to set in a setting process so as to form a poorly soluble solid when brought into contact with an aqueous solution or after being introduced into an aqueous solution.

According to the invention, the structured mineral bone substitute moldings are made of an anhydrous mineral bone cement containing calcium compounds and from 0.5 mol. % to 25 mol. % strontium compounds, based on the total content of divalent cations, or magnesium compounds and from 0.5 mol. % to 25 mol. % strontium compounds, based on the total content of divalent cations, or calcium compounds and magnesium compounds and from 0.5 mol. % to 25 mol. % strontium compounds, based on the total content of divalent cations.

The structured mineral bone substitute moldings are preferably made of an anhydrous mineral bone cement containing from 2 mol. % to 20 mol. % strontium ions, based on the total content of divalent cations, and particularly preferably from 5 mol. % to 15 mol. % strontium ions.

Advantageously, the bone growth on the structured mineral bone substitute moldings is improved by the strontium ions, and the mechanical properties, in particular the compressive strength, of the structured mineral bone substitute moldings are improved by the strontium ions.

In one embodiment, the structured mineral bone substitute molding has residual hydraulic activity.

Hydraulic activity within the meaning of the invention denotes the qualitative capability of a structured mineral bone substitute molding according to the invention to undergo a chemical reaction, i.e. to set, as a result of the addition of water and/or a hydrous component.

In another embodiment, the structured mineral bone substitute moldings having residual hydraulic activity contain from 5 wt. % to 90 wt. %, particularly preferably from 10 wt. % to 80 wt. %, and most particularly preferably from 30 wt. % to 70 wt. % of set mineral bone cement.

The content of set mineral bone cement can be determined by means of X-ray diffractometry (XRD). The Rietveld method can be used for quantification. The Rietveld method is used for quantitative phase analysis, which is to say the quantitative determination of the crystalline components of a powdery sample.

In one embodiment, the setting process or the chemical reaction with water and/or a hydrous component is interrupted by removing water before the reactive mineral bone cement components have fully reacted.

In another embodiment, the residual reactive mineral bone cement component is hydrated by bringing a structured mineral bone substitute molding according to the invention, which has residual hydraulic activity, into contact with water and/or a hydrous component, whereby the interrupted setting process is continued.

In another embodiment, the renewed contact between a structured mineral bone substitute molding according to the invention, which has residual hydraulic activity, and water and/or a hydrous component does not take place until immediately before or during or after use thereof, i.e. during or after implantation into the body.

Advantageously, the structured mineral bone substitute moldings having residual hydraulic activity have a high level of biological activity. According to the invention, “biological activity” denotes the ability to form a crystal structure similar to bone during hardening under biological conditions, whereby interaction with the organic and cellular elements of the new bone being formed is possible. The greater the residual hydraulic activity at the time of use, the greater the biological activity is. A structured mineral bone substitute molding having a predetermined compressive strength and biological activity can thus be provided in a manner matched to the requirements of the particular clinical application.

In one embodiment, the structured mineral bone substitute molding is fully hardened or fully set, i.e. it has no hydraulic activity. This is the case when comparatively high mechanical strength is desirable.

In one embodiment, the fully set structured mineral bone substitute moldings have compressive strengths of at least 10 MPa.

According to the invention, “compressive strength” denotes the capacity of a material to withstand the action of compressive forces. Compressive strength is the quotient of the breaking load and the cross-sectional area A of a test specimen. It is expressed in the form of force per area (in Pa=N/m²). When the applied compressive stress is greater than the compressive strength of a body, the body is destroyed. Additionally, “compressive strength” is understood to mean the uniaxial compressive strength. Compressive strength can be determined on defined moldings having plane-parallel upper and lower faces on a universal testing machine (Hegewald and Peschke) by way of a load cell with a maximum load of 20 kN and an advancement of 1 mm/min.

In one embodiment, the fully set structured mineral bone substitute moldings have compressive strengths in the range of from 10 to 100 MPa, preferably in the range of from 10 to 50 MPa, and particularly preferably in the range of from 10 to 25 MPa.

In another embodiment, the structured mineral bone substitute molding having residual hydraulic activity has at least 5% of the compressive strength of a fully set bone substitute molding made of the same mineral bone cement components and having the same defined interconnecting pore system and the same predetermined structure. Advantageously, a structured mineral bone substitute molding having residual hydraulic activity and at least 5% of the compressive strength of a fully set bone substitute molding has sufficient dimensional stability to be able to withstand the mechanical loads associated with storage and transport, for example.

The object is furthermore achieved by a method for producing structured mineral bone substitute moldings, comprising the following steps:
a) mixing a reactive mineral bone cement, which comprises calcium and/or magnesium compounds and from 0.5 mol. % to 25 mol. % strontium compounds, based on the total content of divalent cations, with an anhydrous carrier liquid to form a moldable bone cement compound;
b) shaping the bone cement compound into a structured mineral bone substitute molding, which has a predetermined structure and a defined interconnecting pore system, by means of 3D printing,
   wherein the diameter of the strands of bone cement compound is between 200 μm and 1200 μm, preferably between 250 μm and 1000 μm, and particularly preferably between 300 μm and 800 μm, and
   wherein the distance between the strands of bone cement compound is between 200 μm and 1500 μm, preferably between 250 μm and 1000 μm, and particularly preferably between 300 μm and 800 μm;
c) initiating the setting process by bringing the bone substitute molding into contact with an aqueous solution or a steam-saturated environment; and
d) terminating the setting process by the substantial removal of water.

Advantageously, the method according to the invention is used to produce structured mineral bone substitute moldings without a ceramic sintering step, with shaping of said molding taking place under controlled conditions, which moldings are solidified by way of a hydraulic setting process such that sufficient dimensional stability for storage, transport and the respective implantation conditions is ensured, wherein the bone substitute moldings fully harden or are fully hardened prior to, during and/or after implantation.

According to the invention, the reactive mineral bone cement comprises calcium compounds and from 0.5 mol. % to 25 mol. % strontium compounds, based on the total content of divalent cations, or magnesium compounds and from 0.5 mol. % to 25 mol. % strontium compounds, based on the total content of divalent cations, or calcium compounds and magnesium compounds and from 0.5 mol. % to 25 mol. % strontium compounds, based on the total content of divalent cations.

In one embodiment of the method according to the invention, a homogeneous moldable bone cement compound in the form of a pasty dispersion is obtained by thoroughly mixing at least one powdery reactive mineral bone cement component with an anhydrous carrier liquid.

In one embodiment of the method according to the invention, at least one substance from the group consisting of silicates, phosphates, sulfates, carbonates, oxides and/or hydroxides is used in conjunction with calcium ions and strontium ions, or magnesium ions and strontium ions, or calcium ions and magnesium ions and strontium ions as the reactive mineral bone cement.

In another embodiment of the method according to the invention, the strontium compounds are selected from strontium carbonates, strontium oxides, strontium hydroxides and/or strontium phosphates, and the strontium compounds are preferably selected from SrCO₃, SrHPO₄, Sr₂P₂O₇, Sr₂(PO₄)₂ and Sr₅(PO₄)₃OH.

In another embodiment of the method according to the invention, at least one substance selected from calcium phosphates, calcium carbonates, calcium oxides, calcium hydroxides, calcium sulfates, calcium silicates, magnesium phosphates, magnesium carbonates, magnesium hydroxide carbonates, magnesium oxides and analogous compounds containing calcium and magnesium in different molar ratios (in particular calcium magnesium phosphates and calcium magnesium carbonates) is used as the reactive mineral bone cement. Additionally, water-soluble alkali or ammonium phosphates, and alkali or ammonium citrates, alkali or ammonium tartrates or alkali silicates, may be present as reactants or to influence the setting reaction.

In a preferred embodiment of the method according to the invention, at least one substance from the group of phosphates in conjunction with calcium, magnesium or strontium ions, including hydrogen and dihydrogen phosphates, is used as the reactive mineral bone cement.

In another embodiment of the method according to the invention, at least one phosphate in conjunction with calcium ions is used as the reactive mineral bone cement, wherein the molar ratio of calcium to phosphate is at least 1, and the molar ratio of divalent cations to phosphate ions is at least 1.35.

In another embodiment of the method according to the invention, calcium and/or magnesium salts of orthophosphoric acid are used as the reactive mineral bone cement.

In another embodiment of the method according to the invention, mixtures of calcium phosphates and/or magnesium phosphates with carbonates, oxides or hydroxides of calcium, magnesium or strontium, or mixtures of carbonates, oxides or hydroxides of calcium, magnesium or strontium with alkali phosphates (monoalkali, dialkali and trialkali phosphates), monoammonium and diammonium phosphates or alkali silicates are used as the reactive mineral bone cement.

According to the invention, the reactive mineral bone cement further comprises an anhydrous carrier liquid.

A “carrier liquid” is understood to mean an organic carrier liquid in which the reactive mineral bone cement is dispersed, wherein a chemical reaction does not take place. An “anhydrous carrier liquid” is understood to mean an organic carrier liquid having a water content of no more than 0.5% (v/v).

According to the invention, the anhydrous carrier liquid is biocompatible, preferred carrier liquids being those already used in medical products and pharmaceutical products (in particular as auxiliary agents), or substances that occur naturally in the body, approved pharmaceutical auxiliary agents being particularly preferred, in particular those approved for parenteral administration, i.e. sterile preparations intended to be injected, infused or implanted in the human or animal body.

The anhydrous carrier liquid is selected from isopropyl myristate, short-chain and medium-chain triglycerides, and short-chain and medium-chain fatty acid esters of glycol.

“Short-chain triglycerides or fatty acid esters” are understood to mean compounds of fatty acids each having a length of from 2 to 5 carbon atoms. “Medium-chain triglycerides or fatty acid esters” are understood to mean compounds of fatty acids each having a length of from 6 to 14 carbon atoms.

The anhydrous carrier liquid is preferably selected from short-chain or medium-chain triglycerides, medium-chain fatty acid esters of ethylene glycol and propylene glycol, polymers of ethylene glycol, short-chain oligomers of propylene glycol, copolymers comprising ethylene glycol and propylene glycol units, polyethylene glycol monomethyl and dimethyl ethers, glycerol and the water-soluble ethers and esters thereof, and diglycerol and polyglycerol.

The anhydrous carrier liquid is particularly preferably selected from esters of triglycerides with fatty acids that, on average, have fewer than 14 carbon atoms, polypropylene glycols, and esters of polypropylene glycols, as well as monoethers and diethers of polypropylene glycols with monoalcohols and polyethylene glycols (PEGs) having a molecular weight of <1000, and the monoethers and diethers thereof.

In another embodiment of the method according to the invention, the percent by weight of anhydrous carrier liquid, based on the total weight of the reactive mineral bone cement, is from 5 wt. % to 25 wt. %, preferably from 5 wt. % to 20 wt. %, and particularly preferably from 5 wt. % to 17.5 wt. %.

In another embodiment of the method according to the invention, the moldable bone cement compound contains additional fillers and/or active ingredients and active pharmacological ingredients, which are not involved in the setting reaction.

In another embodiment of the method according to the invention, the moldable bone cement compound contains surface-active substances, which are selected from the group of surfactants.

In another embodiment of the method according to the invention, the moldable bone cement compound contains at least two surfactants, selected from at least two of the groups of anionic, cationic, amphoteric and non-ionic surfactants.

In a preferred embodiment of the method according to the invention, the moldable bone cement compound contains at least one anionic surfactant and at least one non-ionic surfactant.

In another embodiment of the method according to the invention, the anionic surfactant is selected from fatty acids and the salts thereof, esters of fatty acids and the salts thereof, carboxylic acid ethers, phosphoric acid esters and the salts thereof, acyl amino acids and the salts thereof, preferably from a fatty alcohol esterified with citric acid, sulfuric acid or phosphoric acid, a monoglyceride or diglyceride esterified with citric acid, sulfuric acid or phosphoric acid or the salts thereof, or at least one fatty acid esterified with citric acid, sulfuric acid or phosphoric acid or the salts thereof.

In another embodiment of the method according to the invention, the non-ionic surfactant is selected from fatty alcohols, ethoxylated fatty alcohols, ethylene oxide/propylene oxide block copolymers, ethoxylated fats and oils, polyethylene glycol fatty acid esters, sorbitan esters, ethoxylated sorbitan esters, polyglycerol monoesters and amine oxides, preferably from an ethoxylated fatty alcohol, an ethoxylated fatty acid, an ethoxylated sorbitan fatty acid ester, particularly preferably Polysorbate 80, an ethoxylated fat or an ethoxylated oil, particularly preferably polyethoxylated castor oil.

In another embodiment of the method according to the invention, the total mass of the non-ionic surfactants is at least twice the total mass of the anionic surfactants.

Surface-active substances (surfactants) advantageously promote or enable the penetration of water, steam or atmospheric moisture into the molded bone cement compound or the structured mineral bone substitute moldings having hydraulic activity so as to trigger and advance the setting reaction.

In another embodiment of the method according to the invention, at least one setting accelerator is another component of the reactive mineral bone cement.

Setting accelerators advantageously set the setting time and the progression of the pH value during the setting reaction of the reactive mineral bone cement.

In another embodiment of the method according to the invention, the at least one setting accelerator is selected from phosphate salts, organic acids or salts of organic acids, preferably from phosphates containing sodium and/or potassium ions or ammonium ionsor salts of organic acids containing sodium and/or potassium ions or ammonium ions and mixtures thereof with one another, particularly preferably hydrogen phosphates, most particularly preferably ammonium, sodium or potassium hydrogen phosphate.

In another embodiment of the method according to the invention, the percent by weight of the at least one setting accelerator (based on the weight of the mineral cement powder) in the reactive mineral bone cement according to the invention is from 0.1% to 5%, particularly preferably from 0.2% to 4%, and most particularly preferably from 0.5% to 3.5%.

In another embodiment of the method according to the invention, at least one active pharmacological ingredient is another component of the reactive mineral bone cement.

In another embodiment of the method according to the invention, the active pharmacological ingredients are selected from antibiotics, antiseptics, antimicrobial peptides or antimicrobial proteins, nucleic acids, preferably siRNA; antiresorptive active ingredients, preferably biphosphonates, corticosteroids, fluorides, proton-pump inhibitors; parathormone and the derivatives thereof, bone growth-stimulating active ingredients, preferably growth factors, vitamins, hormones, morphogens, particularly preferably bone morphogenetic proteins and peptides; and angiogenic active ingredients, preferably fibroblast growth factors such as aFGF, bFGF or FGF18; anti-inflammatory agents and anti-tumor substances.

Advantageously, the structured mineral bone substitute moldings are particularly suited as carriers for active pharmacological ingredients, since successive release of the active ingredient is achieved due to the resorption of the mineral bone cement.

In another embodiment of the method according to the invention, at least one filler selected from mineral and organic substances is another component of the reactive mineral bone cement.

In another embodiment of the method according to the invention, the at least one filler is water-soluble. Advantageously, the porosity of the solid formed during the setting process with water is set by using water-soluble fillers.

In another embodiment of the method according to the invention, water-soluble fillers are selected from sugars, preferably sucrose; sugar alcohols, preferably sorbitol, xylitol and mannitol; and water-soluble salts, preferably sodium chloride, sodium carbonate, ammonium carbonate or calcium chloride.

In another embodiment of the method according to the invention, the water-soluble filler has a particle size of from 10 μm to 2000 μm, and preferably from 100 μm to 1000 μm.

In another embodiment of the method according to the invention, the reactive mineral bone cement contains from 5 vol. % to 90 vol. % water-soluble fillers, and preferably from 10 vol. % to 80 vol. %, based on the total volume of the reactive mineral bone cement.

In another embodiment of the method according to the invention, at least one polymeric addition, selected from chitosan, hyaluronic acid, gelatin, collagen, chondroitin sulfate, cellulose derivatives, starch derivatives, alginate, water-soluble acrylates, polyethylene glycol, polyethylene oxide, PEG/PPG copolymers, polyvinylpyrrolidone, and copolymers of water-soluble acrylates with polyethylene glycol and/or polyethylene oxide, is another component of the reactive mineral bone cement.

In another embodiment of the method according to the invention, all the components of the reactive mineral bone cement are resorbable.

According to the invention, the bone cement compound is shaped into a structured mineral bone substitute molding by way of 3D printing. “3D printing” is understood to mean an extrusion-based printing method in which pasty or highly viscous materials are output through dosing needles and deposited as strands in consecutive layers (as stacks) to form three-dimensional objects.

The bone cement compound is shaped into a structured mineral bone substitute molding by way of 3D printing using a computer-aided design (CAD)/computer-aided manufacturing (CAM) process.

A “CAD/CAM process” is understood to mean the combination of computer-aided design, i.e. using a computer to draft and design a virtual model of three-dimensional objects by means of computer-aided manufacturing.

Advantageously, a structured mineral bone substitute molding having a predetermined structure and a defined interconnecting pore system is produced in a highly flexible manner by way of 3D printing.

In one embodiment, the shaping of the bone cement compound by way of 3D printing into defined patterns or a defined interconnecting pore system takes place by varying the position of the strands, the distances between the strands, the orientation of the layers relative to one another (angles), and by depositing several layers of identical strands.

In another embodiment, the bone cement compound is shaped by depositing from 4 to 1000 layers, preferably from 10 to 500 layers, and most particularly preferably from 10 to 100 layers.

In another embodiment, the bone cement compound is shaped by depositing layers oriented at an angle of between 0° and 90° in relation to the layer underneath.

In another embodiment, the orientation of the layers is changed after every layer or every other layer.

In another embodiment, the angle at which the layers are oriented with respect to one another is changed by between 0° and 90°, preferably 15°, 45°, 60° or 90°.

In another embodiment, strands are alternately deposited and oriented at an angle of 90° in relation to the layer underneath.

In another embodiment, strands are alternately deposited so as to be oriented at an angle of 90° in relation to the layer underneath in at least 50 vol. % of the structured mineral bone substitute molding.

In another embodiment, holes are made for fastening elements, with no strands being deposited in these areas, or with strands being deposited around the hole, and optionally further strands being deposited so as to reinforce the material.

Advantageously, osseointegration that is as rapid and complete as possible is achieved through the interconnecting pore system, since the distance that the bone has to grow in is minimal due to the rectilinear pore system.

According to the invention, the setting process is initiated by bringing the bone substitute molding into contact with an aqueous solution or a saturated steam atmosphere.

In another embodiment, the setting process is initiated in a saturated steam atmosphere at a relative humidity of at least 90%.

In another embodiment, the setting process is initiated at a temperature of between 0° C. and 150° C., preferably between 25° C. and 75° C.

In another embodiment, the setting process takes place in several stages, by initiating the setting process in a saturated steam atmosphere and continuing the setting process in at least one further step in at least one aqueous solution. The steps may follow one another directly or with a time interval therebetween.

In another embodiment, the aqueous solution for initiating or continuing the setting process contains at least one additive selected from a buffer solution, an organic and/or inorganic salt, a protein, a cell preparation, a biological, recombinant or pharmacological active ingredient, a nucleic acid, a mixture of nucleic acids, an amino acid, a modified amino acid, a vitamin and a mixture thereof.

In another embodiment, the setting process takes place in a saturated steam atmosphere at a relative humidity of at least 90% for 12 hours to 7 days, and preferably 18 hours to 4 days. In another embodiment, the setting process takes place at a temperature of between 20° C. and 80° C., preferably 37° C. to 50° C.

In another embodiment, at least part of the setting process takes place at a higher temperature (>100° C.) and under increased pressure (>1 bar) in the autoclave and simultaneously serves to sterilize the structured mineral bone substitute molding.

In one embodiment, the setting process is terminated by the substantial removal of water at a temperature of between 0° C. and 150° C. using a drying process, or by replacing the water with at least one volatile, toxicologically safe solvent, preferably selected from acetone and lower alcohols, most particularly preferably acetone.

According to the invention, “water”, which can be substantially removed, is understood to mean physically bound or condensed water.

“Lower alcohols” are understood to mean saturated hydrocarbons comprising a hydroxyl group (OH group), which contain no more than 10 carbon atoms (C atoms), such as methanol, ethanol, isopropanol or propanol. The lower alcohol is preferably ethanol.

Advantageously, the volatile, toxicologically safe solvent is miscible with water and is able to dissolve the anhydrous carrier liquid. Advantageously, replacing the water with at least one volatile, toxicologically safe solvent substantially removes water and completely removes the anhydrous carrier liquid and other auxiliary agents, wherein a person skilled in the art will select the volatile, toxicologically safe solvent in keeping with the solubility properties of the anhydrous carrier liquid used and the auxiliary agents. This does not preclude that some of the carrier liquid and/or auxiliary agents will remain in the structured mineral bone substitute molding according to the invention. The use of biocompatible substances as carrier liquids and auxiliary agents advantageously makes it possible to use such structured mineral bone substitute moldings in the human body.

In another embodiment, the substantial removal of water as a result of the replacement of the water with at least one volatile, toxicologically safe solvent is followed by a drying process for removing any auxiliary agents contained therein, residual water and the solvent.

In one embodiment, the setting process is terminated by a drying process, wherein physical ambient parameters are varied, preferably by decreasing the pressure and/or raising the temperature, or by freeze drying.

In another embodiment, water is substantially removed by being used up during the chemical setting reaction, wherein the present amount of physically bound water is entirely converted into chemically bound water. In this embodiment, less water is provided during the initiation of the setting process than would be required for the entire setting reaction.

In another embodiment, the substantial removal of water is interrupted before the setting reaction is complete, whereby a structured mineral bone substitute molding having residual hydraulic activity is formed.

Advantageously, the structured mineral bone substitute moldings according to the invention are plastically deformable prior to the first time that they are brought into contact with an aqueous solution or a saturated steam atmosphere, but the structured mineral bone substitute moldings having residual hydraulic activity are dimensionally stable, whereby the bone substitute molding having residual hydraulic activity has sufficient mechanical strength for the transportation, storage and implantation thereof. The hardness and compressive strength of the bone substitute moldings having residual hydraulic activity can still be significantly lower than that of a fully set bone substitute molding that is made of the same mineral bone cement components and has the same defined interconnecting pore system and the same predetermined structure, whereby the shaping and/or structuring of an implant on the basis of a bone substitute molding according to the invention in order to interlockingly adapt said implant to the defect of the bone is advantageously simplified for the end user (such as the surgeon), for example by carving using surgical instruments (such as a scalpel).

In another embodiment, complete hardening of the structured mineral bone substitute molding having residual hydraulic activity takes place—after the initiation and termination of the setting process—by again bringing it into contact with an aqueous liquid containing biological components and/or active pharmacological ingredients and/or isolated or cultivated cells.

Advantageously, the method according to the invention for producing structured mineral bone substitute moldings takes place under moderate conditions (temperatures of between 0° C. and 150° C., preferably between 25° C. and 75° C.), whereby the structured mineral bone substitute moldings are particularly suitable as carriers for active pharmacological ingredients since temperature-sensitive active pharmacological ingredients, such as peptides and proteins, nucleic acids or cell preparations, are preserved. Furthermore, unforeseeable reactions between calcium phosphates and strontium compounds are advantageously prevented as a result of these moderate conditions.

Advantageously, high dimensional accuracy of the moldings is achieved by the method according to the invention, wherein neither strand fractures nor surface changes occur in the individual strands.

Another aspect of the invention relates to the use of the structured mineral bone substitute molding according to the invention for producing an alloplastic implant.

An “alloplastic implant” is understood to mean an implant made of synthetic materials foreign to the body.

In another embodiment, the structured mineral bone substitute molding according to the invention is used as an alloplastic implant material for filling bone defects. Bone defects may be congenital or occur as a result of accidents, after surgical procedures, in particular the removal of bone tumors, cysts, prosthesis replacements, corrective osteotomies, etc.

In a preferred embodiment, the structured mineral bone substitute moldings according to the invention are used in oral/maxillary surgery for new bone augmentation, such as alveolar ridge augmentation, sinus lift or for filling voids left by extractions.

In another preferred embodiment, the structured mineral bone substitute moldings according to the invention are used in orthopedics, trauma surgery and spinal column surgery for filling bone defects of all kinds.

Compared to other biological and alloplastic bone substitute moldings currently used, the structured mineral bone substitute moldings according to the invention advantageously have a defined porosity, increased bioactivity, defined mechanical properties and easy intraoperative workability and can be effectively combined with biological substances, bone cells and active pharmacological ingredients.

In another embodiment, the structured mineral bone substitute moldings according to the invention are used as a carrier material in cell culturing, preferably for the cultivation of bone cells, tissue culturing and/or tissue engineering.

“Tissue engineering” is understood to mean the construction of tissue or the cultivation of tissue, in which biological tissue is to be synthetically produced by a directed cultivation of cells.

In another embodiment, the structured mineral bone substitute moldings according to the invention are used as carrier materials in biotechnology, preferably for the cultivation of bacterial or yeast cells, which can be introduced into the carrier material as early as during the manufacturing process.

In another embodiment, the structured mineral bone substitute moldings according to the invention are used as carrier materials for tissue engineering, wherein the structured mineral bone substitute molding according to the invention is colonized in vitro with bone cells, preferably autologous bone cells or stem cells, and incubated under sterile conditions, whereby the cells can multiply. Upon conclusion of the cultivation process, an autologized bone substitute implant is obtained, which is indicated in particular for the regeneration of major bone defects.

“Autologous” is understood to mean as “belonging to the same individual.”

In another embodiment, the structured mineral bone substitute moldings according to the invention are used as a carrier material for tissue engineering in a bioreactor.

Advantageously, the structured mineral bone substitute moldings according to the invention have good prerequisites for this use, in particular—in addition to the above-mentioned properties—variable shaping without any technological size restriction while, at the same time, exhibiting a fully interconnecting pore system, which is a crucial prerequisite for effective perfusion for the culture medium in the bioreactor.

Another application is the purification of (waste) water to remove heavy meals and organic substances.

It is also expedient to combine the above-described embodiments and features of the claims in order to implement the invention.

The invention is to be described in greater detail hereinafter on the basis of several embodiments and the related figures. The embodiments are intended to describe the invention without limiting it.

### BRIEF DESCRIPTION OF THE DRAWINGS

In the drawings:

FIG. 1 shows a cylindrical and a wedge-shaped bone substitute molding; and

FIG. 2 shows the result of the statistical histomorphometic evaluation of the osseointegration of structured mineral bone substitute moldings (reference example) and structured mineral bone substitute moldings comprising strontium compounds after an implantation period of 6 months.

### DETAILED DESCRIPTION OF THE PREFERRED EMBODIMENTS

### Production of the Structured Mineral Bone Substitute Moldings

Mixing a Reactive Mineral Bone Cement without Strontium Compounds (Reference Example):

The reactive mineral bone cement was produced by mixing 60 wt. % α-tricalcium phosphate (α-Ca₃(PO₄)₂), 26 wt. % calcium hydrogen phosphate (monetite, CaHPO₄), 10 wt. % calcium carbonate (CaCO₃), and 4 wt. % precipitated hydroxylapatite (Ca₁₀(PO₄)₆(OH)₂), adding 2.5 wt. % finely ground potassium hydrogen phosphate (K₂HPO₄), and by preparing a paste having a short-chain/medium-chain triglyceride (Miglyol 812, Casar and Lorenz, Hilden, Germany), Cremophor ELP (BASF, Ludwigshafen, Germany) and hexadecyl phosphate (Cetyl Phosphate, Amphisol A, Brenntag AG, MUhlheim an der Ruhr, Germany) and having a powder to liquid ratio of 6:1.

### Mixing a Reactive Mineral Bone Cement Comprising a Strontium Compound:

The reactive mineral bone cement was produced by mixing 60 wt. % α-tricalcium phosphate (α-Ca₃(PO₄)₂), 26 wt. % calcium hydrogen phosphate (monetite, CaHPO₄), 10 wt. % strontium carbonate (SrCO₃), and 4 wt. % precipitated hydroxylapatite (Ca₁₀(PO₄)₆(OH)₂), adding 2.5 wt. % finely ground potassium hydrogen phosphate (K₂HPO₄), and by preparing a paste having a short-chain/medium-chain triglyceride Miglyol 812, Cremophor ELP and hexadecyl phosphate and having a powder to liquid ratio of 6:1.

### Shaping the Bone Cement Compound:

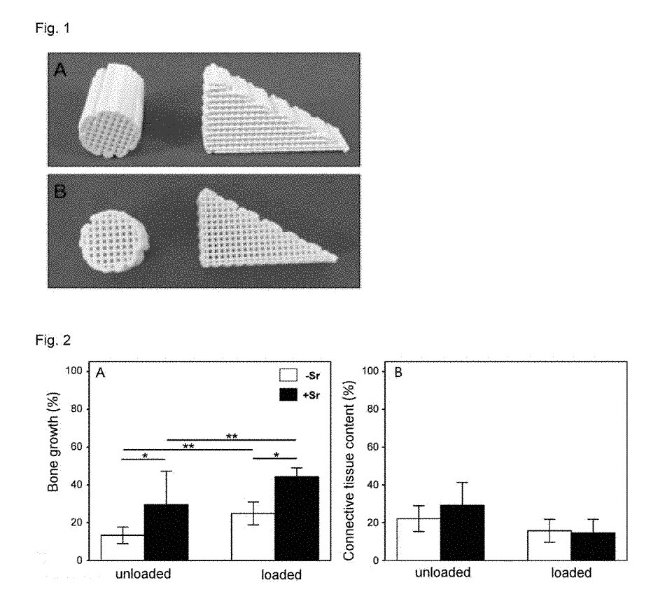

The moldable bone cement compound or the reactive mineral bone cement was poured into 10-ml printing cartridges having a 0.41-mm dosing needle (Nordson, Dettingen unter Teck, Germany). A Discovery 3D printer (regenHU, Villaz-St-Pierre, Switzerland) was used to produce the structured mineral bone substitute moldings. The design of the bone substitute molding is based on the meandering guidance of the dosing needle, and thus the strand deposition, for creating the outline. The three-dimensional expansion was created by alternating orthogonal layers, wherein cylindrical (diameter of 9.2 mm, height of 15 mm) and wedge-shaped (orthogonal dimensions of 24 mm and 14 mm, height 6 mm) bone substitute moldings were produced (FIG. 1). The strand diameter corresponds to the diameter of the dosing needle. The pore system was established by strand distances of 0.59 mm.

FIG. 1 shows the accurate strand deposition and the defined interconnecting pore system in a lateral view (A) and a plan view (B) of the bone substitute molding.

### Setting Process:

After the bone cement compound was shaped, it was incubated in a saturated steam atmosphere having a relative humidity of 95% at 50° C. for 4 days, and subsequently incubated in 0.9% saline solution at 37° C. for 14 days to produce a fully hardened structured mineral bone substitute molding.

### Termination of the Setting Process:

The setting process was terminated by washing the hardened bone substitute moldings three times with acetone (99.5%) and subsequently drying them at 50° C. in an oven under atmospheric pressure.

### Characterization of the Structured Mineral Bone Substitute Moldings

### Determination of the Content of Set Mineral Bone Cement by Way of X-Ray Diffractometry (XRD):

The degree of the reaction can be determined based on the content of α-TCP determined by way of X-ray diffraction. After full hardening, no peak is apparent for α-TCP in the X-ray diffractogram. The peaks for calcite (CaCO₃) and monetite (CaHPO₄) are likewise no longer detectable or can only be identified faintly.

### Determination of the Macroporosity:

The macroporosity is calculated on the basis of the density (CPC paste approximately 2.0 g/cm³) in relation to the weight and volume of the moldings.

The macroporosity of the structured mineral bone substitute moldings is 50% at a density of the moldings of 1.0 g/cm³.

### Determination of the Specific Surface Area by Way of BET Measurement:

The specific surface area of structured mineral bone substitute moldings was determined by way of the gas absorption method according to Brunauer-Emmet-Teller (BET method). Porous moldings measuring 20 mm×10 mm×10 mm and having a strand thickness of 0.33 mm were produced analogously to the above-described method for shaping and subsequent treatment. The moldings were then comminuted and placed as samples in the BET measuring system. The measurement takes place fully automatically. The results are output in the form of a log:

Specific surface area according to BET Sample 1: 11.2238 m²/g
Specific surface area according to BET Sample 2: 12.4238 m²/g
Mean value: 11.8238 m²/g

### Determination of the Compressive Strength:

To determine the compressive strength of the bone substitute moldings according to the invention, a universal testing machine such as that from Hegewald & Peschke was utilized, using a load cell with 20 kN and an advancement speed of 1 mm/min.

The compressive strength of the structured mineral bone substitute moldings without strontium compounds is 10 MPa (reference example), and that of the structured mineral bone substitute moldings according to the invention having strontium compounds is 18 MPa.

### In Vivo Experiments:

The animal study was authorized by the local ethics committee (Tübingen Regional Board, Germany, No. 1142) and agrees with the international recommendations regarding the care and use of laboratory animals (ARRIVE guidelines and EU Directive 2010/63/EU for animal experiments). In total, 14 female Merino sheep (3 to 5 years, 70 to 110 kg) were used for the study. Every sheep received both a structured mineral bone substitute molding without strontium compounds (reference example), and a structured mineral bone substitute molding according to the invention with strontium compounds, each in the hind limbs. The bone substitute moldings were implanted into a drill defect in the distal femoral condyle (unloaded conditions) and in the tibial metaphysis (loaded conditions). Empty defects were omitted since it was already demonstrated that both defect models have a critical size and do not heal during the examination (Kanter et al. 2016; Harms et al. 2012), and to thereby reduce the number of animals tested. After 6 and 26 weeks the sheep were euthanized (n=7 per group), and the femur and the tibia were removed and prepared for histological and energy dispersive X-ray spectroscopy (EDX) analysis.

### Surgical Procedure:

All surgical procedures were carried out under general anesthetic. The sheep received intramuscular pretreatment with 0.2 mg/kg xylazine hydrochloride (Rompun®, 2%, Bayer Leverkusen, Germany). The anesthetic was induced by way of intravenous injection of 5.0 mg/kg thiopental (Thiopental Inresa, Inresa GmbH, Freiburg, Germany) and maintained by way of isoflurane inhalation (Forene®, Abbott GmbH, Wiesbaden, Germany). The sheep were subcutaneously administered 4.0 mg/kg carprofen (Rimadyl®, Pfizer, Karlsruhe, Germany) for analgesia, and 7.0 mg/kg amoxicillin trihydrate (Veyxyl®, VeyxPharma GmbH, Schwarzenborn, Germany) for antibiotic prophylaxis for 4 days.

The structured mineral bone substitute moldings without strontium compounds (reference example) and the structured mineral bone substitute moldings according to the invention with strontium compounds were implanted into the right and left hind limbs, respectively. The surgical procedure was carried out in keeping with Kanter et al. or Harms et al. (Kanter et al. 2016; Harms et al. 2012), wherein a defect having a diameter of 10 mm and a depth of 15 mm was drilled into the trabecular bone of the femur, and a wedge-shaped defect (width of 14 mm, length of 24 mm, height of 6 mm) was produced in the trabecular bone 3 mm beneath the central tibial plateau. Bone fragments were carefully cleaned from the defects by rinsing with sterile saline solution, and the bone substitute moldings were inserted.

To label newly formed bone, tetracycline hydrochloride (25 mg/kg body weight) and calcein green (10 mg/kg body weight) were injected 2 and 4 weeks, respectively, after implantation for sheep that were euthanized after 6 weeks, and 8 and 16 weeks, respectively, after implantation for sheep euthanized after 26 weeks.

### Histological Analyses:

The implants and approximately 10 mm of the surrounding bone were removed and prepared for non-decalcified histology. The samples were fixed in 4%-buffered formaldehyde solution, dehydrated in a series of solutions having an increasing ethanol content, and embedded in methacrylate (Merck KGaA, Darmstadt, Germany). 70 μm sections were prepared and stained with paragon (Paragon C&C, New York, N.Y., US) in accordance with standard protocols. The samples were examined under the light microscope (Leica DMI6000B, MMAF software Version 1.4.0 MetaMorph®, Leica, Heerbrugg, Switzerland) using 50-fold magnification and evaluated in a blind test. Photoshop (Adobe Systems, Mountain View, Calif., US) and MATLAB® (MATLAB R2009b, TheMathsWorks Inc., Natick, Mass., US) were used for image analysis. The surface area of the bone substitute molding of the bone and of the connective tissue, as well as the relative implant surface area in direct contact with the bone, were determined.

Osteoclasts were identified by histologic staining of the tartrate-resistant acid phosphatase (TRAP), following decalcification of the samples and embedding in paraffin in accordance with Recknagel et al. (Recknagel et al. 2013). Cells having at least 3 nuclei, which are located on the bone or the bone substitute molding and showed positive TRAP stains, were defined as osteoclasts. The cells were counted in 6 visible areas (approximately 8 mm²) in the center on the implant using 100-fold magnification. The spatial distribution of the tetracycline- and calcein-labeled bone was analyzed under fluorescent light for the qualitative determination of the temporal bone growth.

### Energy Dispersive X-Ray Spectroscopy (EDX):

The strontium content was determined by grinding the methacrylate-embedded samples and coating them with carbon (MED 010, Balzers Union, Dietenheim, Germany).

The EDX analysis was carried out using a Zeiss DSM 962 scanning electron microscope (Zeiss, Jena, Germany) and Genesis Line/Map 6.44 software (EDAX, Weiterstadt, Germany). The spectral maps were recorded using an accelerating voltage of 20 kV.

### Statistics:

Differences between the 6-week and 26-week groups were identified by using the non-parametric Mann-Whitney U test. To determine the differences within an implantation period, the significance was determined by way of the Wilcoxon signed-rank test. The statistics was carried out by way of SPSS software (v21, SPSS Inc., Chicago, Ill., US). The significance level was established at p 0.05.

FIG. 2 shows the result of the statistical histomorphometic evaluation of the osseointegration of structured mineral bone substitute moldings (reference example) and structured mineral bone substitute moldings comprising strontium compounds after an implantation period of 6 months: new bone growth (A) and connective tissue content (B) in structured mineral bone substitute moldings (reference example) (white bars), and structured mineral bone substitute moldings with strontium compounds (black bars) under loaded and unloaded conditions. *, **) respective significant difference p<0.05.

### Cited Non-Patent Literature (Only Background Material)

M. P. Hofmann, U. Gbureck, C. O. Duncan, M. S. Dover, J. E. Barralet (2007) Carvable calcium phosphate bone substitute material. J. Biomed. Mater. Res. B Appl. Biomater., 83B, 1, 1-8.
A. Lode, K. Meissner, Y. Luo, F. Sonntag, S. Glorius, B. Nies, C. Vater, F. Despang, T. Hanke, M. Gelinsky (2014) Fabrication of porous scaffolds by three-dimensional plotting of a pasty calcium phosphate bone cement under mild conditions. J. Tissue Eng. Regen. Med., 8, 682-693.
E. W. Washburn (1921) The Dynamics of Capillary Flow. Physical Review Band, 17, No. 3, 273-283.
B. Kanter, M. Geffers, A. Ignatius, U. Gbureck (2016) Control of in vivo mineral bone cement degradation. Acta Biomater., 10, 3279-3287.
C. Harms, K. Helms, T. Taschner, I. Stratos, A. Ignatius, T. Gerber et al. (2012) Osteogenic capacity of nanocrystalline bone cement in a weight-bearing defect at the ovine tibial metaphysis. Int. J. Nanomedicine. 7, 2883-2889.
S. Recknagel, R. Bindl, C. Brochhausen, M. Gockelmann, T. Wehner, P. Schoengraf et al. (2013) Systemic inflammation induced by a thoracic trauma alters the cellular composition of the early fracture callus. J. Trauma. Acute Care Surg., 74, 531-537.

1. A structured mineral bone substitute moldings having a defined interconnecting pore system and a predetermined structure,
made of an anhydrous mineral bone cement containing calcium and/or magnesium compounds and from 0.5 mol. % to 25 mol. % strontium ions, based on the total content of divalent cations,
wherein the defined interconnecting pore system, in at least one dimension, has an interconnecting pore system having pores that have an average pore cross section of at least 50,000 μm² and an average pore diameter of at least 250 μm, based on a circular cross section, wherein the defined interconnecting pore system, in at least one dimension, has an interconnecting pore system having pores that have a maximum average pore cross section of 785,000 μm² and a maximum average pore diameter of 1000 μm, based on a circular cross-section.

2. The structured mineral bone substitute moldings as per claim 1, characterized in that the structured mineral bone substitute moldings have a total porosity of between 50% and 85%.

3. The structured mineral bone substitute moldings as per claim 1, characterized in that the structured mineral bone substitute moldings have a specific surface area of at least 5 m²/g.

4. The structured mineral bone substitute moldings as per claim 1, characterized in that the structured mineral bone substitute moldings comprise from 2 mol. % to 20 mol. % strontium ions, based on the total content of divalent cations.

5. The structured mineral bone substitute moldings as per claim 1, characterized in that the defined interconnecting pore system, in at least one dimension, has an interconnecting pore system having pores that have a maximum average pore cross section of 785,000 μm² and a maximum average pore diameter of 1000 μm, based on a circular cross section.

6. A method for producing structured mineral bone substitute moldings, comprising the following steps:
a. mixing a reactive mineral bone cement, which comprises calcium and/or magnesium compounds and from 0.5 mol. % to 25 mol. % strontium compounds, based on the total content of divalent cations, with an anhydrous carrier liquid to form a moldable bone cement compound;
b. shaping the bone cement compound into a structured mineral bone substitute molding, which has a predetermined structure and a defined interconnecting pore system, by means of 3D printing,
wherein the diameter of the strands of bone cement compound is between 200 μm and 1200 μm, and
wherein the distance between the strands of bone cement compound is between 250 μm and 1000 μm;
c. initiating the setting process by bringing the bone substitute molding into contact with an aqueous solution or a saturated steam atmosphere; and
d. terminating the setting process by the substantial removal of water.

7. The method as per claim 6, characterized in that at least one substance from the group consisting of silicates, phosphates, sulfates, carbonates, oxides and/or hydroxides is used in conjunction with calcium ions and/or magnesium ions and strontium ions as the reactive mineral bone cement.

8. The method as per claim 6, characterized in that at least one phosphate in conjunction with calcium ions is used as the reactive mineral bone cement, the molar ratio of calcium to phosphate being at least 1, and the molar ratio of divalent cations to phosphate ions being at least 1.35.

9. The method as per claim 6, characterized in that the aqueous solution for initiating the setting process contains at least one additive selected from a buffer solution, an organic and/or inorganic salt, a protein, a cell preparation, a biological, recombinant or pharmacological active ingredient, a nucleic acid, a mixture of nucleic acids, an amino acid, a modified amino acid, a vitamin and a mixture thereof.

10. The method as per claim 6, characterized in that the setting process is initiated in a saturated steam atmosphere at a relative humidity of at least 90% and a temperature of between 0° C. and 150° C.

11. The method as per claim 6, characterized in that the setting process is terminated by the substantial removal of water using a drying process, or by replacing the water with at least one volatile, toxicologically safe solvent.

12. The use of a structured mineral bone substitute molding according to claim 1 for producing an alloplastic implant.

13. The use of a structured mineral bone substitute molding according to claim 1 as a carrier material in cell culturing, tissue culturing and/or in tissue engineering.
